(19) United States
(12) Patent Application Publication
(10) Pub. No.: US 2014/0369583 A1
(43) Pub. Date: Dec. 18, 2014

(54) ULTRASOUND DIAGNOSTIC DEVICE, ULTRASOUND DIAGNOSTIC METHOD, AND COMPUTER-READABLE MEDIUM HAVING RECORDED PROGRAM THEREIN

(71) Applicant: KONICA MINOLTA, INC., Tokyo (JP)

(72) Inventors: Bumpei TOJI, Hashima-shi (JP); Jun OHMIYA, Kyotanabe-shi (JP); Tadamasa TOMA, Kadoma-shi (JP)

(21) Appl. No.: 14/303,341

(22) Filed: Jun. 12, 2014

(30) Foreign Application Priority Data
Jun. 18, 2013 (JP) 2013-127258

(51) Int. Cl. G06T 7/00; A61B 8/06; A61B 8/08

(52) U.S. Cl. CPC G06T 7/0012; A61B 8/5292; A61B 8/06; G06T 2207/10132; G06T 2207/30101; G06T 2207/30104
USPC 382/131

(57) ABSTRACT

An ultrasound diagnostic device processes ultrasound images obtained by ultrasound scanning on a diagnostic target. The ultrasound images include a set of material images for analyzing shape of the diagnostic target and a diagnostic image for analyzing a diagnostic value of a specific part of the diagnostic target. An image analysis measurement unit generates a schematic diagram showing schematic shape of the diagnostic target based on the shape of the diagnostic target. A relative position calculation unit calculates a relative position of local shape of the specific part to the shape of the diagnostic target. A diagnostic value superimposing unit identifies a position on the schematic shape of the diagnostic target that corresponds to a position of the specific part based on the relative position, and superimposes data of the diagnostic value on the identified position on the schematic shape of the diagnostic target shown in the schematic diagram.

This application is based on an application No. 2013-127258 filed in Japan on Jun. 18, 2013, the contents of which are hereby incorporated by reference.

### BACKGROUND OF THE INVENTION

(1) Field of the Invention

The present invention relates to an ultrasound diagnostic device and a diagnostic position identification method.

(2) Description of the Related Art

According to the ultrasound diagnostic device and the diagnostic position identification method, ultrasound is transmitted to a subject by an ultrasound probe, and ultrasound reflected from the subject is received thereby to obtain ultrasound images in accordance with a reflected ultrasound signal. Medical workers observe such ultrasound images to diagnose whether arteriosclerosis and vascular disease exist.

Known diagnostic targets of ultrasound diagnostic devices include intima-media thickness (hereinafter, referred to as IMT) and plaque. IMT indicates thickness of intima and media of vascular walls. Plaque indicates an elevated lesion which is a localized projection of an inner wall of a blood vessel towards an inner side (lumen) of the blood vessel. Plaque assumes various forms such as thrombus, fatty, fibrous, and so on, and might be a cause for narrowing and occlusion of a carotid artery as well as cerebral infarction and cerebral ischemia. Generally, it is considered that arteriosclerosis advances throughout the entire body, and accordingly a superficial carotid artery is the main target for measurement in determining severity of arteriosclerosis. Therefore, it is inevitable to diagnose whether plaque exists on a superficial carotid artery in order to early discover arteriosclerosis.

### SUMMARY OF THE INVENTION

An ultrasound image of a blood vessel is a tomographic image obtained by representing, in an image, ultrasound echo data resulting from decoding a reflected ultrasound signal. Since one tomographic image covers only several centimeters range of the blood vessel, a patient having no expert knowledge cannot determine a position of a plaque part of the blood vessel only by seeing such an ultrasound image, and cannot take diagnostic results as an objective truth. The same applies to the case where the patient is presented with a list of diagnostic values.

In response to this problem, enrichment of examination report in ultrasound diagnosis is improved by including, in an examination report, a schema of a carotid artery into which a position of a plaque, IMT, and so on are additionally written. However, there is a problem that a diagnostic part is not precisely represented because the plaque drawn in an examination report even by medical workers is often in handwriting with rough brush strokes. On the other hand, it is a great burden for medical workers, who are under pressure of daily tasks, to precisely draw the diagnostic part for the schema included in the examination report.

The present invention aims to provide an ultrasound diagnostic device capable of simply creating examination reports with a proper reliability.

The above aim is achieved by an ultrasound diagnostic device that analyzes a plurality of ultrasound images that are obtained by ultrasound scanning on a diagnostic target, the ultrasound images including a set of material images for analyzing a shape of the diagnostic target and a diagnostic image for analyzing a diagnostic value of a specific part included in the diagnostic target, the ultrasound diagnostic device comprising: a generation circuit that generates a schematic diagram showing a schematic shape of the diagnostic target based on the shape of the diagnostic target that is analyzed from the material images; a calculation circuit that calculates a relative position of a local shape of the specific part shown in the diagnostic image with respect to the shape of the diagnostic target analyzed from the material images; and a superimposing circuit that identifies a position on the schematic shape of the diagnostic target shown in the schematic diagram that corresponds to a position of the specific part based on the relative position, and superimposes data of the diagnostic value on the identified position on the schematic shape of the diagnostic target shown in the schematic diagram.

### BRIEF DESCRIPTION OF THE DRAWINGS

These and the other objects, advantages and features of the invention will become apparent from the following description thereof taken in conjunction with the accompanying drawings which illustrate a specific embodiment of the invention.

In the drawings:

FIG. 1 shows an outer appearance structure of an ultrasound diagnostic device 150 relating to Embodiment 1;

FIG. 2A schematically shows a situation in which a medical worker linearly moves an ultrasound probe on a subject to sequentially obtain reflected ultrasound, FIG. 2B shows a first process of ultrasound scanning, and FIG. 2C shows a second process of ultrasound scanning;

FIG. 3A shows a schematic diagram in which data of a diagnostic value is superimposed on a computer graphics, and FIG. 3B shows a schematic diagram in which the data of the diagnostic value is superimposed on a schema;

FIG. 4 is a block diagram showing an internal structure of the ultrasound diagnostic device 150 relating to Embodiment 1;

FIG. 5 shows a data flow between a B-mode image generation unit 104, an image analysis measurement unit 105, a schematic diagram generation unit 106, a relative position calculation unit 107, and a measured data superimposing unit 108;

FIG. 6 shows a set of transverse images that are obtained by scanning within a 6 cm range and a longitudinal image that is obtained by ultrasound scanning in a longitudinal direction;

FIG. 7A shows a set of transverse images, FIG. 7B shows an example of a schema, FIG. 7C shows a process of searching for a relative position using areas under graphs, and FIG. 7D shows overlapping of a longitudinal image graph with the schema;

FIG. 8 shows a relationship between a real space where a display unit 111 exists and a 3D virtual space for CG generation;

FIG. 9A shows a cross-section of a 3D geometric model on a position of a base point, and FIG. 9B shows a plurality of contour lines of a lumen-intima interface that are extracted from the 3D geometric model;

FIG. 10A shows a contour line lint of the lumen-intima interface that passes through pixels P1, P11, P21, P31, P41, . . . on the transverse images, and FIG. 10B shows an example of change information corresponding to the contour line lint of the lumen-intima interface;

FIG. 11A shows a contour line of a lumen-intima interface analyzed from a set of transverse images and a contour line of the lumen-intima interface shown in a longitudinal image, FIG. 11B shows comparison between a Y-shape base point graph and a longitudinal image graph, and FIG. 11C shows a situation in which the longitudinal image graph is placed on coordinates Rz, and the longitudinal image graph is overlapped with a 3D geometric model;

FIG. 12 is a flow chart showing a processing procedure of examination report creation;

FIG. 13 is a flow chart showing a processing procedure of schematic diagram generation;

FIG. 14 is a flow chart showing a processing procedure of relative position calculation in the longitudinal direction;

FIG. 15 is a flow chart showing the processing procedure of relative position calculation in the longitudinal direction, continuing from FIG. 14;

FIG. 16 is a flow chart showing a processing procedure of superimposed image generation;

FIG. 17 is a block diagram showing an internal structure of an ultrasound diagnostic device 152 relating to Embodiment 2;

FIG. 18 is a flow chart showing a processing procedure of examination report creation in Embodiment 2; and

FIG. 19 is a flow chart showing a processing procedure of relative position calculation based on a 3D geometric model in the longitudinal direction in Embodiment 3.

### DESCRIPTION OF THE PREFERRED EMBODIMENTS

In relation to the ultrasound diagnostic device disclosed in the Background Art section, the inventors have found the following problem.

In order to more efficiently create examination reports, there is a method of introducing report creation support software or the like to a workstation to create examination reports using the software or the like. In this case, it is general that the shape of a plaque is drawn and comments are written via input equipment such as a mouse, a tablet, and a keyboard. However, an operator needs to operate the workstation separately from an ultrasound diagnostic device for diagnose, and this is troublesome for the operator. Furthermore, there is a case where such an operation rather than handwriting places a burden on the operator. In response to this problem, the inventors considered to implement an ultrasound diagnostic device having a function of automatically creating examination reports (auto report function).

Such an ultrasound diagnostic device records a position on a diagnostic target to which each of captured ultrasound images corresponds to. The inventors considered to use a position sensor for obtaining such a corresponding position in an ultrasound image. However, in order to increase the reliability of the position in the ultrasound image to be recorded, it is necessary to use a high-precision position sensor, and this causes the cost increase. For this reason, the inventors abandoned the use of such a position sensor.

Since handwriting in the schema included in the examination report is performed for the purpose of patients' understanding, a high precision is unnecessary and there is a need for an ultrasound diagnostic device capable of simply and easily creating examination reports. Conventionally, there has been no ultrasound diagnostic device capable of simply and promptly creating examination reports including a diagram which is easy for patients to understand.

In view of this, the inventors decided to implement an ultrasound diagnostic device capable of further simply creating examination reports without introducing a high-precision position sensor.

The following describes embodiments of an ultrasound diagnostic device capable of simply creating examination reports, with reference to the drawings. Here, the generation circuit, the calculation circuit, and the superimposing circuit, which are the constituent elements of the ultrasound diagnostic device, each may be constituted from a hardware circuit such as an ASIC (Application Specific Integrated Circuit) and an FPGA (Field Programmable Gate Array), or each may be constituted from a program, for example.

Each of the embodiments described below shows a general or specific example. The numerical values, shapes, materials, structural elements, the arrangement and connection of the structural elements, steps, the processing order of the steps etc. shown in the following embodiments are mere examples, and therefore do not limit the scope of the present invention. Also, structural elements that are not recited in any one of the independent claims among the structural elements in the following embodiments are described as arbitrary structural elements.

### Embodiment 1

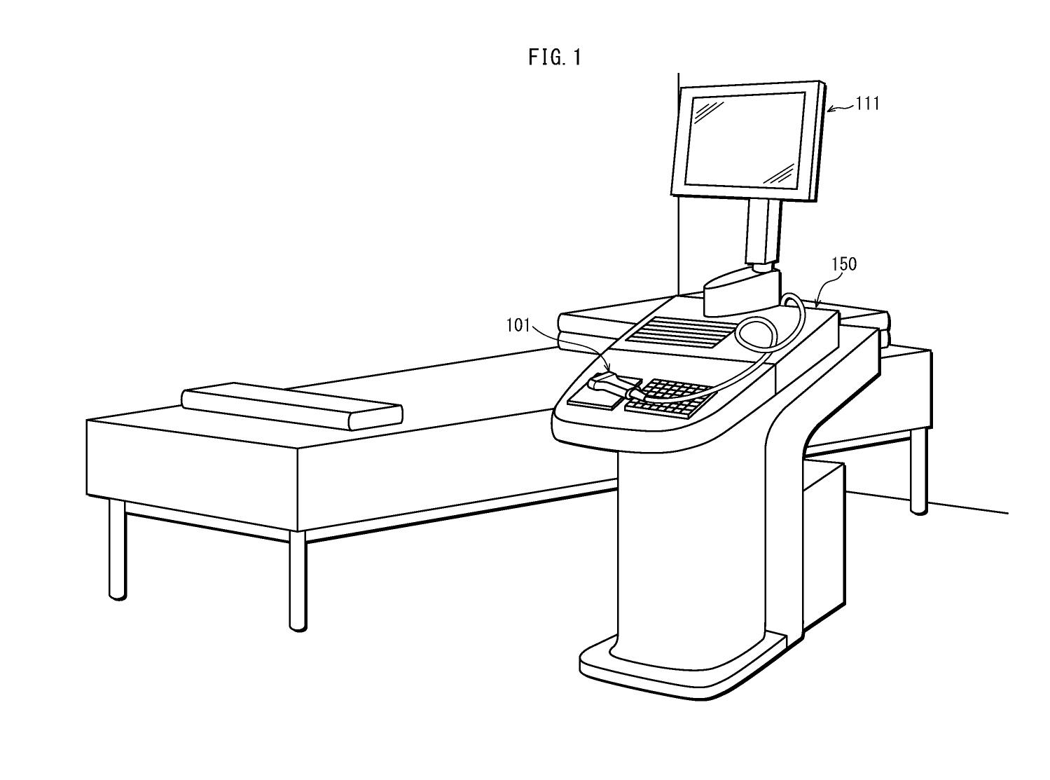

The following describes a schematic structure of an ultrasound diagnostic device relating to the present embodiment. FIG. 1 shows an outer appearance structure of an ultrasound diagnostic device 150 relating to the present embodiment. As shown in the figure, the ultrasound diagnostic device 150 is medical equipment used in medical practice, and is composed of a probe 101 and a display unit 111 that are integrated into one device.

The probe 101 transmits ultrasound, and receives an ultrasound signal reflected from a subject.

The display unit 111 is an LCD (Liquid Crystal Display) or the like, and displays B-mode images, a schematic diagram, diagnostic values, and so on that are generated from the reflected ultrasound signal.

The ultrasound diagnostic device 150 is a processing device that provides a diagnostic imaging function and a network communication function in order to realize ultrasound diagnosis. The diagnostic imaging function is a function of forming an ultrasound image such as a B-mode image based on a reflected ultrasound signal received by the probe 101 and analyzing the ultrasound images to obtain a diagnostic value of a specific diagnosed part to realize processing based on the diagnostic value. The network communication function is a function for, via a network, printing an examination report that is created based on the diagnostic value, storing such an examination report in a server device as data, and so on. In addition, the ultrasound diagnostic device 150 includes a pointing device for designating an arbitrary part on a screen of the display unit 111 and a keyboard for inputting texts of an examination report.

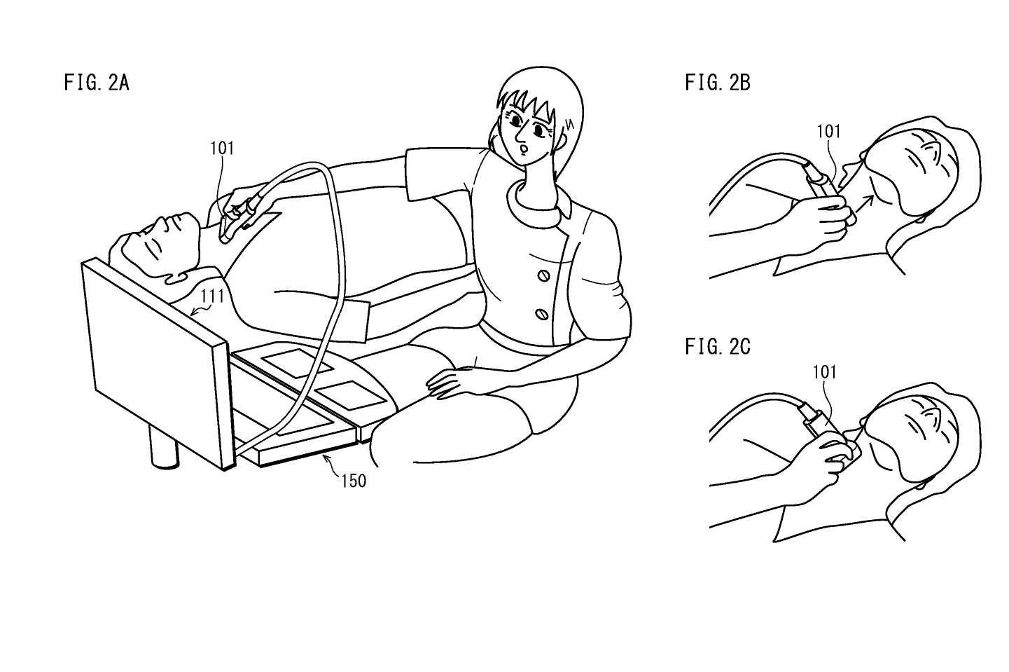

The following describes the use of the ultrasound diagnostic device 150 with reference to FIG. 2. An operator of the ultrasound diagnostic device 150 is a medical worker such as a doctor and a clinical examiner, and makes a diagnosis by applying the probe 101 against a subject to transmit ultrasound to the subject.

Results of the diagnosis are displayed on the display unit 111 in real time. FIG. 2A schematically shows a situation in which a medical worker linearly moves the probe 101 on a subject to sequentially obtain reflected ultrasound.

Ultrasound scanning by the probe 101 is performed through two processes. FIG. 2B shows a first process of ultrasound scanning. In the first process, transverse scanning is performed in which an operator applies the probe 101 against a patient's neck in the transverse direction, and unidirectionally moves the probe 101 in the longitudinal direction of the patient's carotid artery, such that a cross-section perpendicular to the longitudinal direction of the carotid artery is drawn. As a result of this movement, B-mode images are obtained, each of which represent a cross-section perpendicular to the longitudinal direction of a target blood vessel (hereinafter, referred to as transverse images). A set of transverse images correspond to a set of material images in the claims Although depending on the operators' skill, a scanning speed of such an ultrasound probe is around 0.5 cm/second, and an approximately 6 cm range is scanned. As a result, 256 transverse images are obtained. The respective transverse images are identified by frame numbers of 0 to 255. Note that the scanning range and the number of transverse images differ for each examination. The above numerical values of 6 cm and 256 images are just examples.

FIG. 2C shows a second process of ultrasound scanning. In the second process, the operator applies the probe 101 against the patient's throat in the longitudinal direction to transmit ultrasound to the patient's neck. Through this process, a B-mode image is obtained which represents a cross-section parallel to the longitudinal direction of the target blood vessel (hereinafter, referred to as a longitudinal image). The longitudinal image corresponds to a diagnostic image in the claims. Note that B-mode images may be sequentially generated in a time series. The longitudinal image is obtained for the following reasons. Since IMT and so on need to be measured in detail and a B-mode image itself needs to be measured, it is necessary to generate a longitudinal image which covers a large range of a blood vessel. Also, the academic society encourages measurement of the IMT using a longitudinal image.

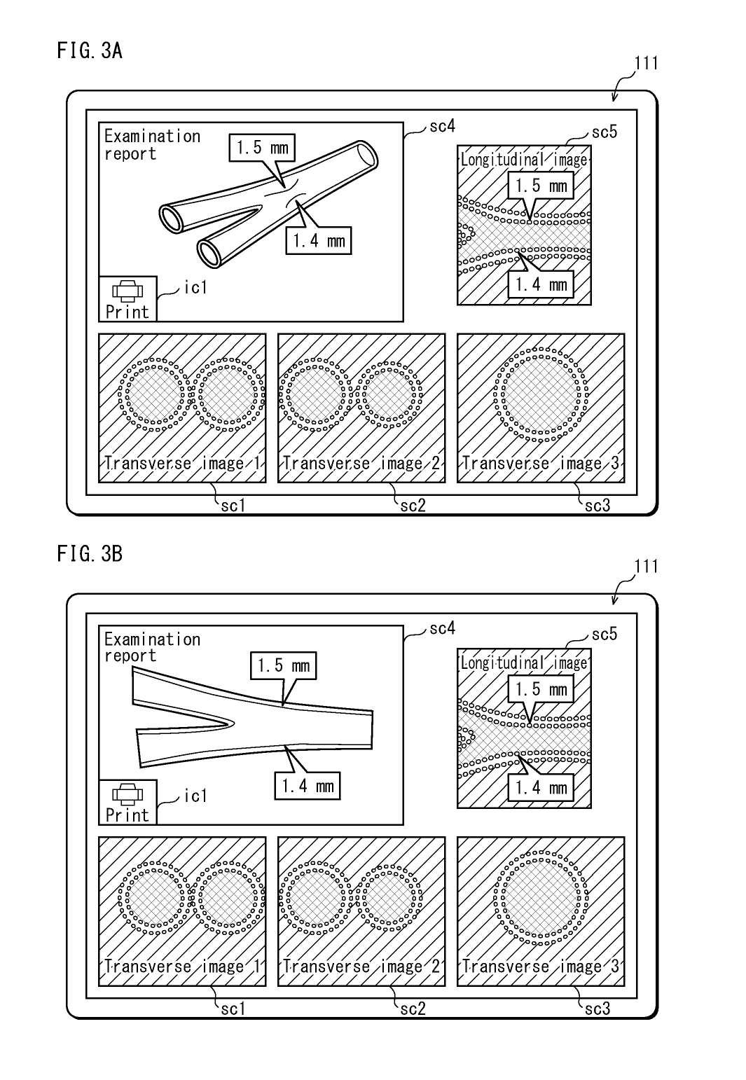

While operations by the probe 101 are performed, the display unit 111 displays a longitudinal image, transverse images, and a schematic diagram, which are generated by ultrasound scanning. FIG. 3A shows an example of contents displayed by the display unit 111. As shown in the figure, the display unit 111 has the screen including three windows sc1, sc 2, and sc 3 for displaying transverse images, a window sc4 for displaying a schematic diagram, and a window for displaying a longitudinal image, for example. The window sc4 for displaying a schematic diagram has a print icon ic1. Operations of this icon ic1 allow a schematic diagram displayed by the display unit 111 to be printed on a sheet. In this way, the display unit 111 displays the longitudinal image, the transverse images, and the schematic diagram to provide a multifaceted perspective of a status of a diagnostic target of a subject.

There are two types of schematic diagrams. One is a schematic diagram in which data of a diagnostic value is superimposed on a computer graphics (CG) of a blood vessel. The other is a schematic diagram in which data of a diagnostic value is superimposed on a schema showing a cross-section of a blood vessel. FIG. 3A shows an image of a schematic diagram in which data of a diagnostic value is superimposed on a CG, and FIG. 3B shows an image of a schematic diagram in which data of a diagnostic value is superimposed on a schema. An operator can switch the display settings of each of the windows shown in FIG. 3A and FIG. 3B by using the pointing device included in the ultrasound diagnostic device 150. Specifically, the operator can switch the display setting of the window sc4 for displaying a schematic diagram between schema and CG. Also, the operator can display the schematic diagram on full screen by deleting the transverse images and the longitudinal image. While probe operations such as shown in FIG. 2A to FIG. 2C are performed, a schema or CG schematic diagram is displayed by the display unit 111 to the user as shown in FIG. 3A or FIG. 3B. This allows prompt judgment as to whether any failure occurs in display of the schema or CG schematic diagram. This completes the description of the screen configuration of the ultrasound diagnostic device 150. The following describes the structure of the ultrasound diagnostic device 150 relating to the present embodiment.

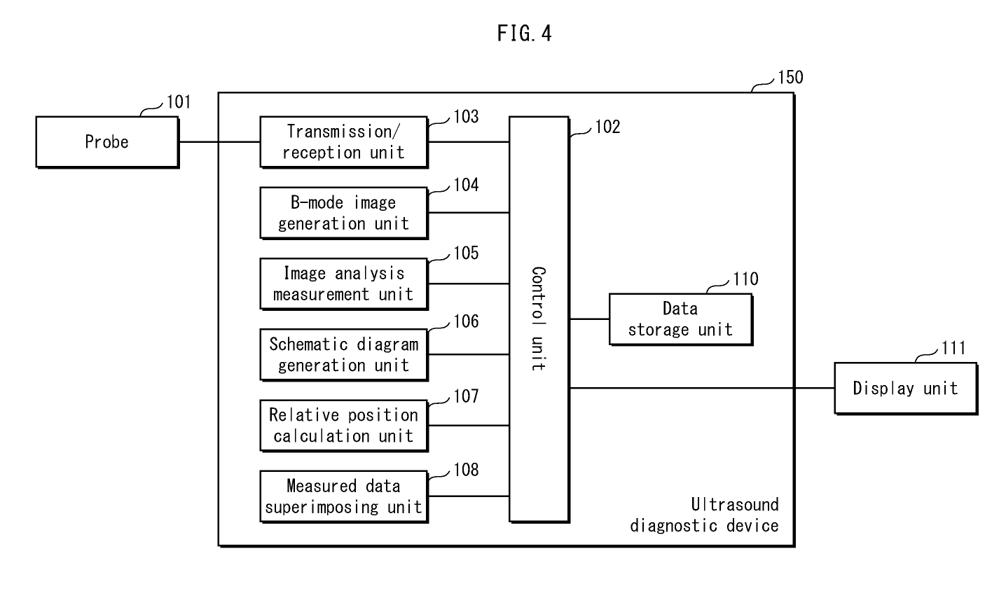

FIG. 4 is a block diagram showing an internal structure of the ultrasound diagnostic device 150 relating to the present embodiment. The ultrasound diagnostic device 150 includes, as shown in FIG. 4, a control unit 102, a transmission/reception unit 103, a B-mode image generation unit 104, an image analysis measurement unit 105, a schematic diagram generation unit 106, a relative position calculation unit 107, a measured data superimposing unit 108, and a data storage unit 110. Also, the probe 101 and the display unit 111 are provided outside the ultrasound diagnostic device 150, and are connected with the ultrasound diagnostic device 150. The constituent elements shown in FIG. 4, namely, the control unit 102, the transmission/reception unit 103, the B-mode image generation unit 104, the image analysis measurement unit 105, the schematic diagram generation unit 106, the relative position calculation unit 107, and the measured data superimposing unit 108, each may be constituted as a single circuit component, or may be constituted as a circuit component assembly of a plurality of circuit components.

The control unit 102 includes a CPU, a ROM, and a RAM, and controls processing units included in the ultrasound diagnostic device 150. Hereinafter, although no special description is given, the control unit 102 controls operations of the processing units. For example, the control unit 102 causes the processing units to perform processing while controlling an operation timing and so on.

The transmission/reception unit 103 drives ultrasound transducers of the probe 101 to generate ultrasound, and receives a reflected ultrasound signal which is received by the probe 101 from a subject.

The B-mode image generation unit 104 performs filter processing on a reflected ultrasound signal, and then performs envelope detection. Furthermore, the B-mode image generation unit 104 performs logarithmic conversion and gain control on a signal resulting from the envelope detection to generate a B-mode image.

With respect to transverse images, the image analysis measurement unit 105 extracts a closed curve representing a contour of a media-adventitia interface and a closed curve representing a contour of a lumen-intima interface, and measures a distance between these contours as IMT. With respect to a longitudinal image, the image analysis measurement unit 105 extracts a straight line or a curved line representing a contour of a media-adventitia interface and a straight line or a curved line representing a contour of a lumen-intima interface, and measures a distance between these contours as IMT.

The schematic diagram generation unit 106 generates a schematic diagram of a target blood vessel based on the IMT measured by the image analysis measurement unit 105 or the transverse images.

The relative position calculation unit 107 calculates a relative position in the schematic diagram based on the transverse images and a diagnostic value analyzed from the longitudinal image. Specifically, the relative position calculation unit 107 searches positions on a shape of a diagnostic target analyzed from the transverse images for a position in which the shape of the diagnostic target is the most similar to a local shape of a diagnosed part included in the diagnostic target shown in the longitudinal image. Then, the relative position calculation unit 107 generates relative position information indicating, as the relative position, the position in which the shape of the diagnostic target is the most similar, which is found as a result of the searching.

The measured data superimposing unit 108 superimposes data of the diagnostic value on a superimposing position in the schematic diagram of the target blood vessel that corresponds to the position of the diagnosed part based on the relative position information calculated by the relative position calculation unit 107 to generate a superimposed image.

The data storage unit 110 stores therein, together with a document file, a file of a B-mode image generated by the B-mode image generation unit 104, a file of a schematic diagram generated by the schematic diagram generation unit 106, a file of analysis results obtained by the image analysis measurement unit 105, and a file of a superimposed image generated by the measured data superimposing unit 108.

The document file is a text of an examination report, which is composed of a combination of transverse images and a longitudinal image. Furthermore, the data storage unit 110 stores therein a data file of a schema model. The schema model is graphic data representing a general shape of a cross-section of a blood vessel, and line width thereof and color therewithin can be freely changed. By changing the line width of the schema model, it is possible to generate various types of schemas each representing a blood vessel differing in IMT.

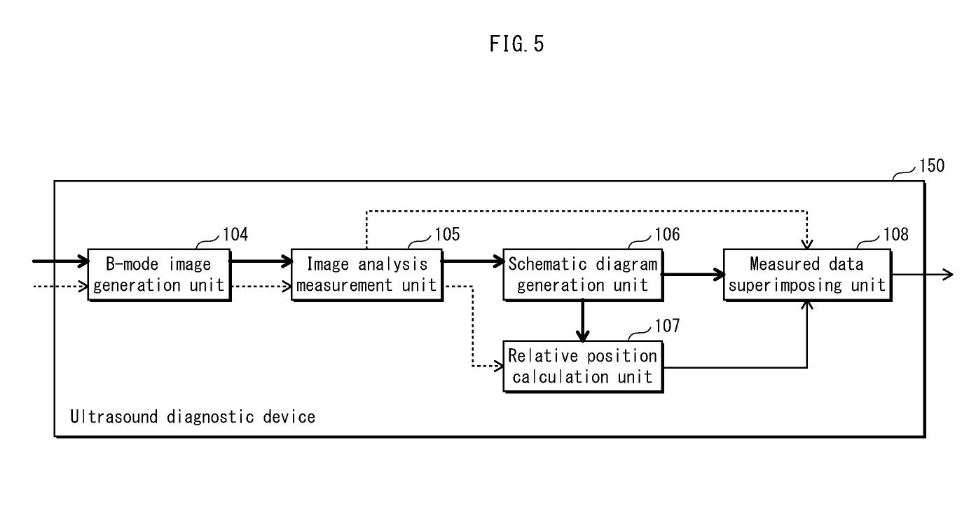

The following describes a data flow showing transfer of B-mode images and relevant information between the constituent elements. FIG. 5 shows a data flow between the B-mode image generation unit 104, the image analysis measurement unit 105, the schematic diagram generation unit 106, the relative position calculation unit 107, and the measured data superimposing unit 108. In FIG. 5, thick lines represent a flow of a set of transverse images and analysis results corresponding thereto, and dashed lines represent a longitudinal image showing a diagnosed part included in a diagnostic target of a target blood vessel and a diagnostic value and analysis results corresponding thereto.

Upon receiving an input of a reflected ultrasound signal or B-mode images from the probe 101, the image analysis measurement unit 105 performs image analysis based on the reflected ultrasound signal or the B-mode images to calculate IMT of a target blood vessel. The IMT, which is analyzed from a set of transverse images, is transferred to the schematic diagram generation unit 106. The diagnostic value, which is analyzed from a longitudinal image, is transferred to the relative position calculation unit 107 and the measured data superimposing unit 108.

The schematic diagram generation unit 106 generates a schematic diagram of the target blood vessel based on the IMT analyzed from the transverse images, and transfers the schematic diagram to the measured data superimposing unit 108.

The relative position calculation unit 107 calculates a relative position of a local shape of the diagnosed part shown in the longitudinal image with respect to a shape of the diagnostic target analyzed from the transverse images based on the diagnostic value analyzed from the longitudinal image, and transfers relative position information indicating the relative position to the measured data superimposing unit 108.

The measured data superimposing unit 108 generates an image in which data of the diagnostic value is superimposed on a superimposing position in the schematic diagram of the target blood vessel that corresponds to the position of the diagnosed part (superimposed image), based on the schematic diagram of the target blood vessel transferred from the schematic diagram generation unit 106, the diagnostic value transferred from the image analysis measurement unit 105, and the relative position calculated by the relative position calculation unit 107.

The following describes, using a specific example, a process of generating a schematic diagram performed by the constituent elements of the ultrasound diagnostic device 150 namely, the image analysis measurement unit 105, the schematic diagram generation unit 106, the relative position calculation unit 107, and the measured data superimposing unit 108. The specific example used here is an example in which a part of a blood vessel where a plaque exists is displayed in a schema schematic diagram.

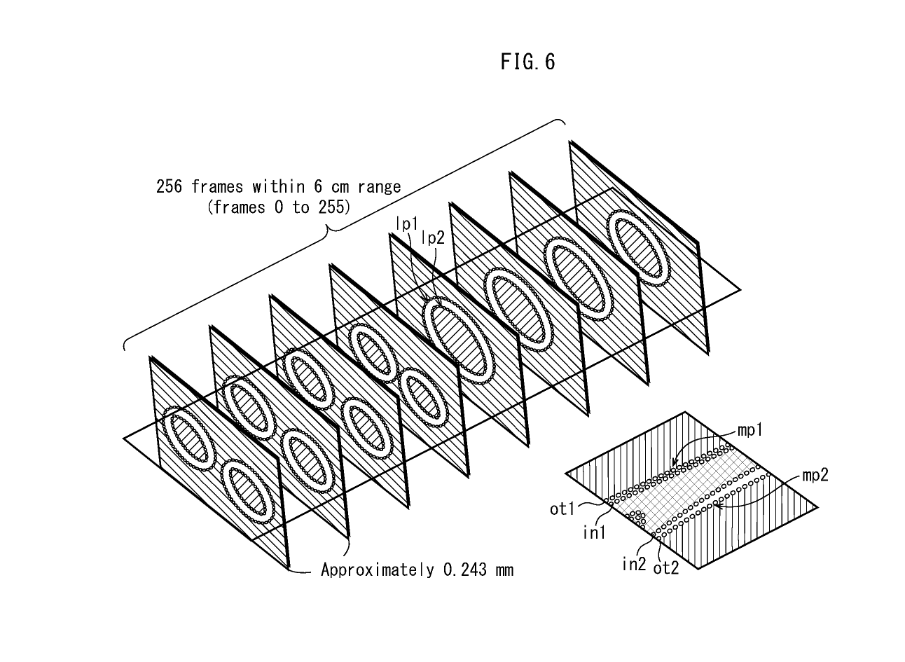

FIG. 6 shows a set of transverse images that are generated by scanning on a 6 cm range and a longitudinal image that is obtained by ultrasound scanning in the longitudinal direction. The transverse images are compared with the longitudinal image. Since the number of the transverse images generated by transverse scanning is 256, a shape of a blood vessel in the 6 cm range is roughly recognized. In the first process, transverse scanning is performed to capture 256 transverse images per 6 cm range, and accordingly the Z-coordinates along the Z-axis are arranged at intervals of 0.243 mm (60 mm/256). The longitudinal image, which is positioned at the front side in the figure, corresponds to one of the transverse images when horizontally placed. Accordingly, a range of the shape that is recognizable from the longitudinal image is an only limited range.

The following describes analysis of a set of transverse images by the image analysis measurement unit 105. In FIG. 6, the transverse images each include double closed curves 1p1 and 1p2. One of the closed curves that is on the external side, namely, the closed curve 1p1 represents a contour of a media-adventitia interface of a vascular wall. The other closed curve that is on the internal side, namely, the closed curve 1p2 represents a contour of a lumen-intima interface of the vascular wall. The image analysis measurement unit 105 performs searching outward in the radial direction from a position P on the inner closed curve 1p2 in each of the transverse images. The image analysis measurement unit 105 obtains, as IMT, a distance resulting from the searching, namely, a distance from a pixel on the internal closed curve 1p2 to a pixel on the external closed curve 1p1 in the radial direction. This completes the description of analysis of transverse images by the image analysis measurement unit 105.

The following describes analysis of longitudinal image by the image analysis measurement unit 105. In FIG. 6, the longitudinal image includes four contour lines in1, in2, ot1, and ot2. The pair of contour lines in1 and in2 on the inner side each represent a contour of the lumen-intima interface of the vascular wall in the longitudinal direction of the blood vessel. The pair of contour lines ot1 and ot2 on the external side each represent a contour of the media-adventitia interface of the vascular wall in the longitudinal direction of the blood vessel. The image analysis measurement unit 105 performs searching toward the external contour lines in the radial direction from a position on the inner contour line shown in the longitudinal image. The image analysis measurement unit 105 obtains, as IMT, a distance resulting from the searching, namely, a distance from the internal contour line to the external contour line. In this way, the image analysis measurement unit 105 identifies the distance in the radial direction as the IMT. Also, the image analysis measurement unit 105 identifies a part having the IMT exceeding a predetermined threshold value as a plaque part. Parts mp1 and mp2 in FIG. 6 each have IMT exceeding the predetermined threshold value, and accordingly are identified as a diagnosed part that is a target for examination report. A diagnostic value, which is obtained by the image analysis measurement unit 105 analyzing the longitudinal image, indicates IMT, blood flow velocity, and elasticity of a vascular tissue, in addition to plaque. This completes the description of analysis of longitudinal image by the image analysis measurement unit 105.

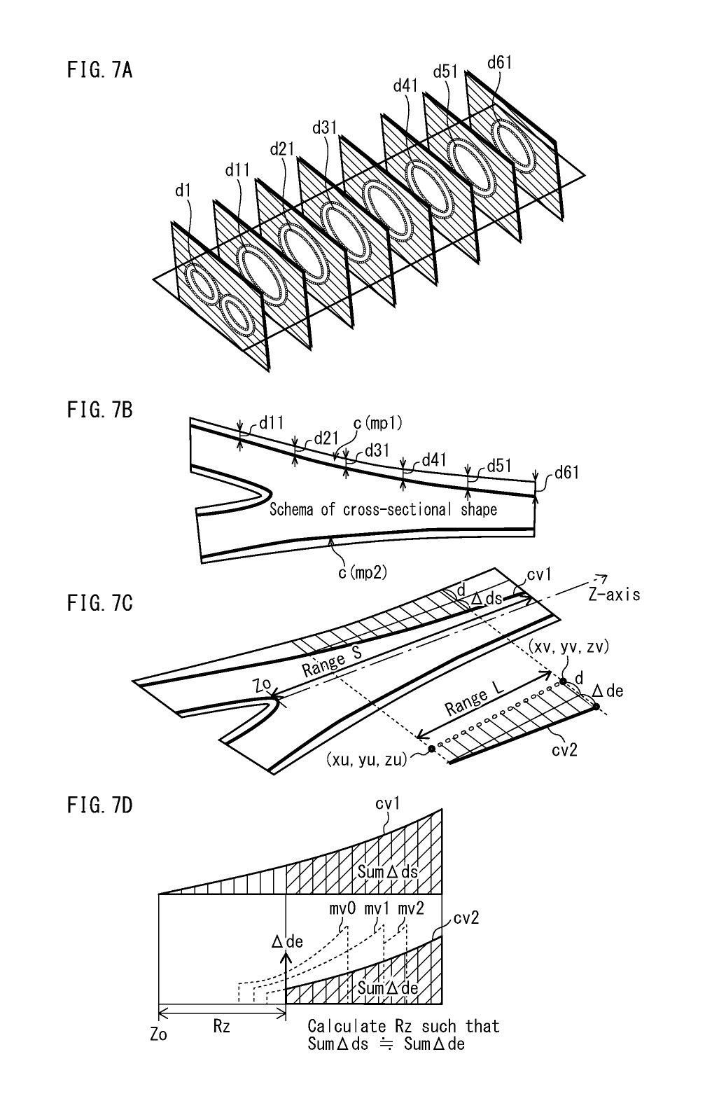

The following describes processing by the schematic diagram generation unit 106. The description is given here on the case where processing is performed on a schema schematic diagram. FIG. 7A shows a set of transverse images. In FIG. 7B, reference signs d1, d11, d21, d31, d41, . . . each indicate IMT analyzed from transverse images by the image analysis measurement unit 105. After the IMT is analyzed by the image analysis measurement unit 105, the schematic diagram generation unit 106 reads a schema model from the data storage unit 110, and scales the schema model in accordance with the IMT analyzed by the image analysis measurement unit 105. FIG. 7B shows an example of scaling of a schema model. In FIG. 7B, arrows relating to the IMTs d1, d11, d21, d31, d41, . . . each indicate scaling, namely, reducing or magnifying, a line width in the schema model by the IMT analyzed from the transverse images. This scaling results in the schema model read from the data storage unit 110 that is similar to an external shape of a target blood vessel.

This completes the description of the processing by the schematic diagram generation unit 106. The following describes in detail processing by the relative position calculation unit 107.

In order to display the diagnosed parts mp1 and mp2, which are analyzed from the longitudinal image, in the schema shown in FIG. 7B, it is necessary to identify a position in the schema that corresponds to each of positions of the diagnosed parts mp1 and mp2 shown in the longitudinal image.

For this reason, the schematic diagram generation unit 106 searches positions on the contour line of the lumen-intima interface shown in the schema for a position in which the contour line of the lumen-intima interface is the most similar to the contour line of the lumen-intima interface shown in the longitudinal image.

When finding the most similar position, the schematic diagram generation unit 106 calculates relative position information indicating a relative position between the shape of the lumen-intima interface shown in the schema and the shape of the contour line of the lumen-intima interface shown in the longitudinal image. In FIG. 7B, reference signs c(mp1) and c(mp2) represent conversion points that are respectively obtained by adding the relative position indicated by the relative position information to the positions of the parts mp1 and mp2 shown in the longitudinal image.

In FIG. 7C, a reference sign Zo represents a Y-shaped bifurcation of the blood vessel shown in the schema. Searching for the most similar position is performed using this Y-shaped bifurcation as an origin of relative coordinates of the Z-coordinates. That is, the carotid artery has a Y-shaped bifurcation that is bifurcated into two parts. Since this Y-shaped bifurcation is noticeable in structure of the blood vessel, the contour line of the lumen-intima interface is extracted using the Z-coordinates in the bifurcation as a base point. In FIG. 7C, a reference sign cv1 represents the contour line of the lumen-intima interface in the schema, which has the Y-shaped bifurcation as the base point, and a reference sign cv2 represents the contour line of the lumen-intima interface in the longitudinal image, which is analyzed from the longitudinal image by the image analysis measurement unit 105. The contour line of the lumen-intima interface shown in the longitudinal image is composed of change Δde in IMT. A sequence of the change Δde is hereinafter referred to as a longitudinal image graph. Also, a sequence of change Δds in an overlapping part in the schema that overlaps with the contour line of the lumen-intima interface shown in longitudinal image is hereinafter referred to as a Y-shape base point graph.

The relative position calculation unit 107 places the contour line of the lumen-intima interface shown in the longitudinal image at various coordinates along the Z-axis starting from the position of the base point to overlap the lumen-intima interface shown in the longitudinal image with the lumen-intima interface shown in the schema. Then, the relative position calculation unit 107 calculates a difference in area in the overlapped position between the longitudinal image graph and the Y-shape base point graph. The relative position calculation unit 107 identifies, from among the overlapped positions, an overlapped position for which the smallest difference in area between the longitudinal image graph and the Y-shape base point graph is calculated. The overlapped position in which the difference in area is the smallest is determined as the relative position because of being considered to be a position in which the contour line of the lumen-intima interface shown in the longitudinal image is the most similar to the contour line of the lumen-intima interface shown in the schema. Range S represents horizontal width in the Z-axis direction in the schema, and Range L represents horizontal width in the Z-axis direction in the longitudinal image graph. In FIG. 7C, the longitudinal image graph is moved within this Range S to search for Z-coordinates of a position in which the difference in area between the longitudinal image graph and the Y-shape base point graph is the smallest.

In FIG. 7D, SumΔde represents area under the longitudinal image graph, and is calculated as the sum of the IMTs belonging to the longitudinal image graph. SumΔds represents area under the Y-shape base point graph in the schema, and is calculated as the sum of the IMTs belonging to the overlapping part. Values mv0, mv1, and mv2 represent transition of the movement of the longitudinal image graph.

A value Rz represents relative Z-coordinates in which the difference in area between the longitudinal image graph and the Y-shape base point graph is the smallest. Since the coordinates Rz are equivalent to offset from the coordinates of the base point in the schema, the respective positions of the diagnostic parts mp1 and mp2 shown in the longitudinal image are respectively converted to the positions c(mp1) and c(mp2) in the schema by adding the coordinates Rz to the Z-coordinates of the diagnostic part shown in the longitudinal image. The measured data superimposing unit 108 superimposes the data of the diagnostic value on the positions c(mp1) and c(mp2). As a result, an examination report including a superimposed image such as shown in FIG. 3B is obtained.

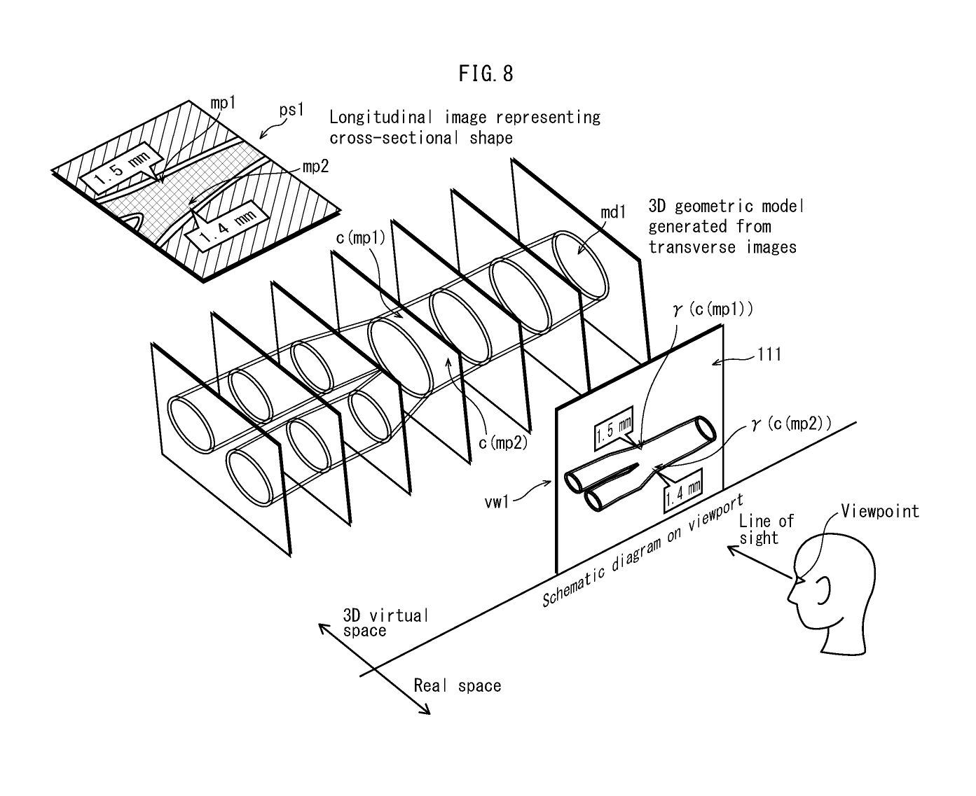

The following describes processing by the image analysis measurement unit 105 in the case where a schematic diagram is a CG schematic diagram. FIG. 8 shows a relationship between a real space where the display unit 111 exists and a 3D virtual space for CG generation. The 3D virtual space is defined using coordinates of a coordinate system in which the longitudinal direction of the blood vessel is determined as the Z-axis direction, and a plane where a cross-section exists is determined as the X-Y plane (global 3D coordinates).

The display unit 111 corresponds to a viewport vw1 in the 3D virtual space. The viewport vw1 is a virtual screen that is placed between a 3D geometric model and a viewpoint position. A projected image to be displayed by the display unit 111 is generated by the viewport vw1.

The image analysis measurement unit 105 generates a 3D geometric model from which a CG is to be generated, based on coordinates of pixels on a closed curve of 256 transverse images. Then, the image analysis measurement unit 105 projects vertex coordinates of the 3D geometric model to the viewport vw1 to obtain the CG from which a schematic diagram is to be generated.

In the figure, reference sign md1 represents a 3D geometric model that is obtained by performing curved surface interpolation between cross-sectional shapes shown in a set of transverse images. The 3D geometric model is a model of a virtual vascular structure that is formed using the 3D coordinates in the 3D virtual space. Vertex coordinates of the 3D geometric model are each composed of an X-coordinate and a Y-coordinate representing the contour line shown in the transverse images and a Z-coordinate representing dimension determined by the frame number. A reference sign ps1 represents a planar shape including a diagnosed part shown in the longitudinal image. Reference signs mp1 and mp2 represent a diagnosed part of a plaque, and reference signs c(mp1) and c(mp2) respectively represent positions in the 3D geometric model corresponding to positions of the diagnosed parts mp1 and mp2. Reference signs γ(c(mp1)) and γ(c(mp2)) represent mapping points that are respectively obtained by mapping the positions c(mp1) and c(mp2) to the viewport vw1. This completes the description of the processing by the image analysis measurement unit 105 in the case where a schematic diagram is a CG schematic diagram. The following describes processing by the relative position calculation unit 107 in the case where a schematic diagram is a CG schematic diagram.

The relative position calculation unit 107 calculates relative position information by searching positions on the 3D geometric model for a position that corresponds to a position of the diagnosed part included in the planar shape shown in the longitudinal image. In order to precisely project the 3D geometric model to the viewport vw1 which is the display unit 111, it is necessary to precisely calculate the positions c(mp1) and c(mp2) on the 3D geometric model, which respectively correspond to the positions of the diagnosed parts mp1 and mp2 shown in the longitudinal image.

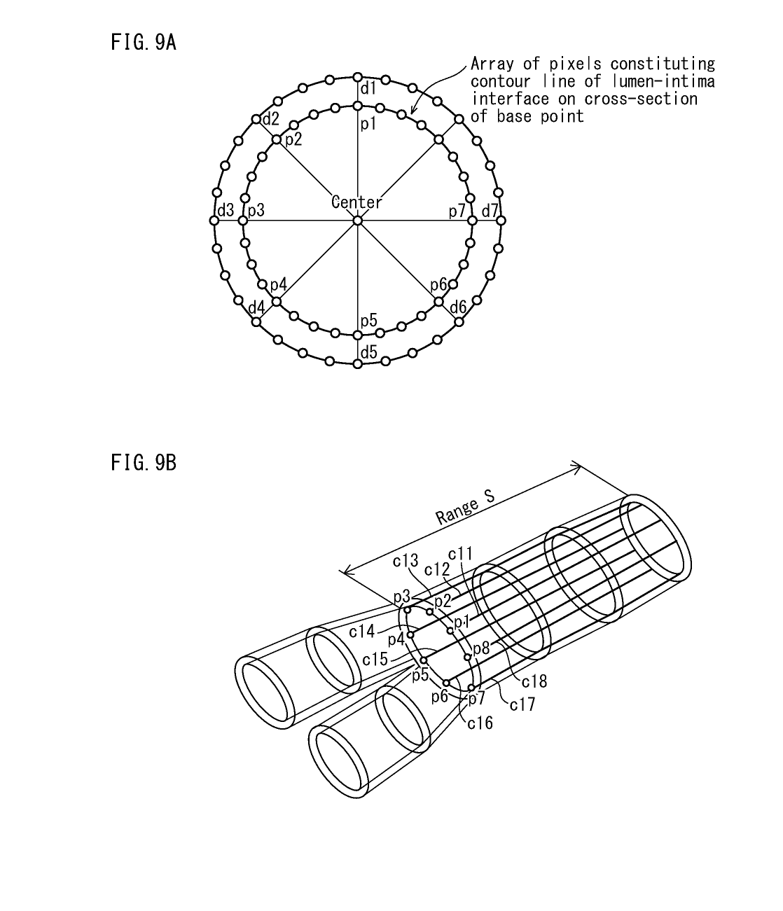

Since the schema is planar, a target for overlapping with the contour line of the lumen-intima interface shown in the longitudinal image is either the contour line of the lumen-intima interface on the upper side in the schema or the contour line of the lumen-intima interface on the lower side in the schema. In the case of the CG schematic diagram compared with this, it is possible to extract a plurality of contour lines of the lumen-intima interface from the 3D geometric model. This is because the blood vessel three-dimensionally expands in the CG schematic diagram. FIG. 9A shows a cross-section of a 3D geometric model on a position of a base point. In FIG. 9A, reference signs P1, P2, P3, P4, . . . represent a plurality of pixels constituting a circumference of a transverse cross-section on the position of the base point, and are each a start point of a change curve representing change in IMT in the longitudinal direction. In FIG. 9B, reference signs c11, c12, c13, c14, . . . represent contour lines of the lumen-intima interface respectively having, as start points, pixels P1, P2, P3, P4, . . . that lie on the circumference of the transverse cross-section on the position of the base point.

In the case of the CG schematic diagram, the contour line of the lumen-intima interface shown in the longitudinal image is overlapped with each of the contour lines of the lumen-intima interface respectively having the start points P1, P2, P3, P4, . . . . A position is searched for, among positions along the Z-axis, in which a difference in area is the smallest in the overlapped position between the longitudinal image graph and the Y-shape base point graph on each of the contour lines of the lumen-intima interface.

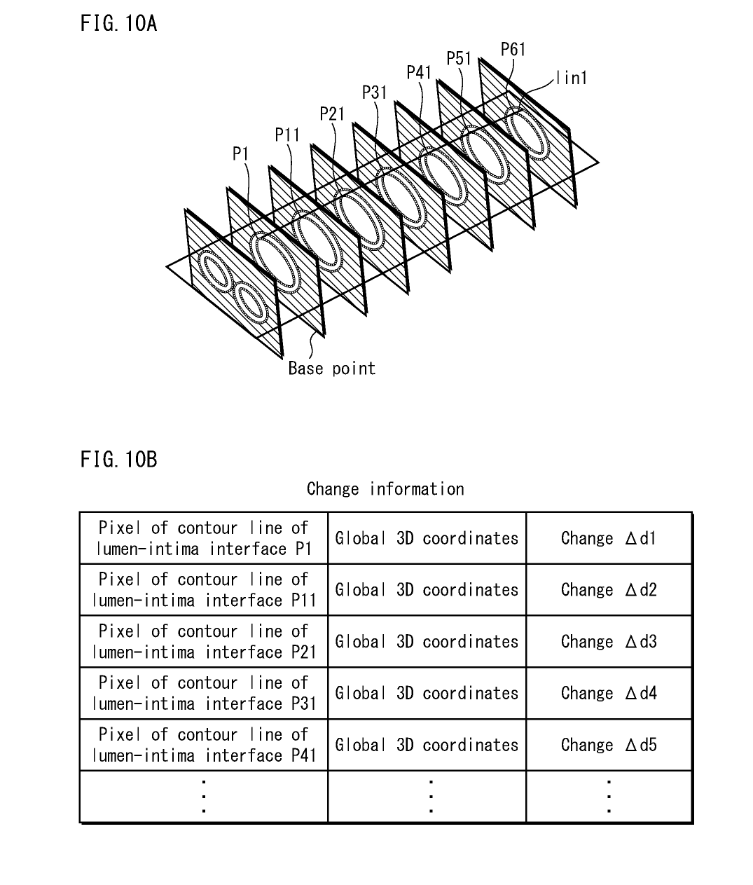

The Y-shape base point graph is defined by the relative position calculation unit 107 generating change information. FIG. 10A shows a contour line lint of the lumen-intima interface that passes through the pixels P1, P11, P21, P31, P41, . . . on the transverse images. FIG. 10B shows an example of change information corresponding to the contour line lint of the lumen-intima interface. As shown in FIG. 10B, the change information indicates respective global 3D coordinates in the 3D virtual space of the pixels P1, P11, P21, P31, P41, . . . on the transverse images in one-to-one correspondence with the change Δd1, Δd2, Δd3, Δd4, . . . in IMT in the contour line of the lumen-intima interface. The longitudinal image graph is also defined by generation of the similar change information. The change information indicates that the global 3D coordinates of the pixels, through which the contour line of the lumen-intima interface passes, are in one-to-one correspondence with the change in IMT. Accordingly, the respective shapes of the Y-shape base point graph and longitudinal image graph are defined by the change information. This completes the description of the processing by the relative position calculation unit 107 in the case where a schematic diagram is a CG schematic diagram.

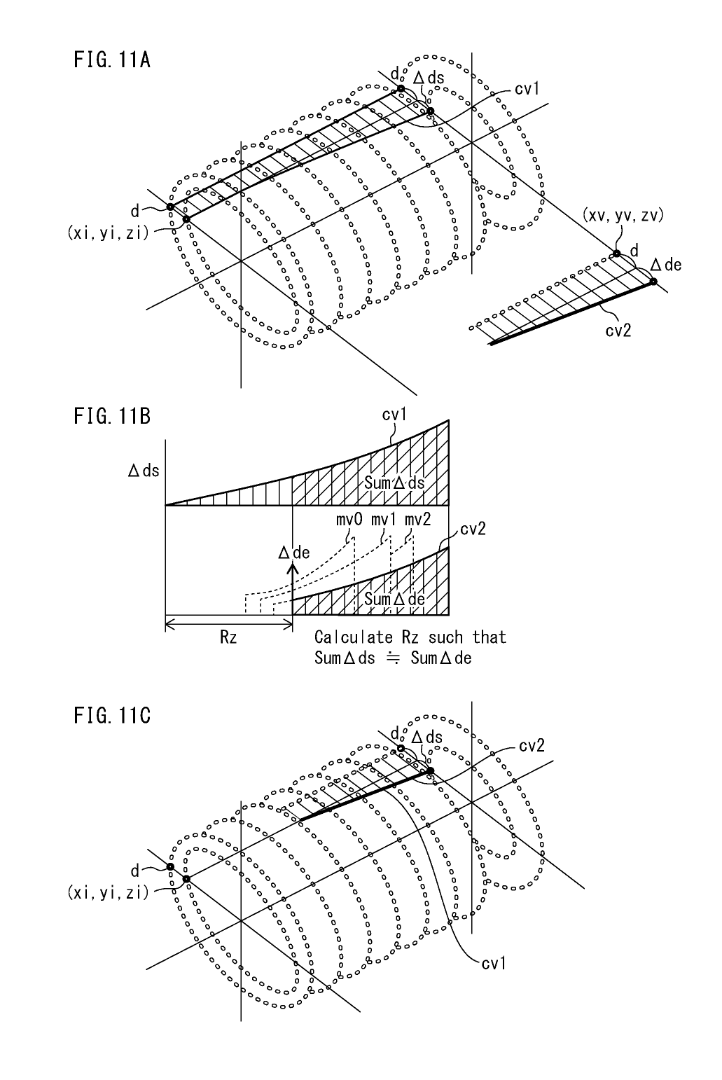

FIG. 11A shows a contour line of a lumen-intima interface analyzed from a set of transverse images and a contour line of the lumen-intima interface shown in a longitudinal image. FIG. 11B shows, in the upper stage, a Y-shape base point graph in which the horizontal axis represents coordinates in the Z-axis direction of a 3D geometric model, and the vertical axis represents change Δds in IMT at coordinates of the transverse images. SumΔds represents area under the Y-shape base point graph. A value Rz represents relative Z-coordinates in which the difference in area between the longitudinal image graph and the Y-shape base point graph is the smallest. FIG. 11B shows, in the lower stage, a longitudinal image graph in which the horizontal axis represents coordinates in the transverse direction in the longitudinal image, and the vertical axis represents change Δde in IMT at coordinates of the longitudinal image. SumΔde represents area under the longitudinal image graph which is the sum of the change Δde.

FIG. 11C shows a situation in which the longitudinal image graph is placed on the coordinates Rz, and the longitudinal image graph is overlapped with the 3D geometric model.

The Z-coordinates are searched for in which a difference in area between the longitudinal image graph and the Y-shape base point graph is the smallest, to thereby obtain the coordinates Rz. The measured data superimposing unit 108 adds the coordinates Rz to the positions of the parts mp1 and mp2 to obtain conversion points c(mp1) and c(mp2), projects the conversion points c(mp1) and c(mp2) to the viewport, and superimposes data of the diagnostic value on the projected points c(mp1) and c(mp2). As a result, an examination report including a superimposed image such as shown in FIG. 3B is obtained.

This completes the description of the specific processing by the constituent elements of the ultrasound diagnostic device 150.

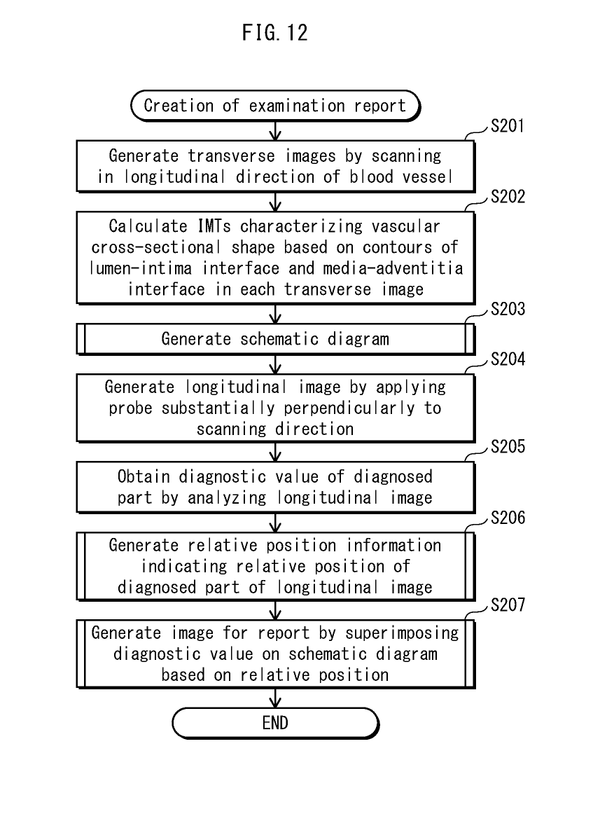

The following describes a processing procedure for causing the constituent elements of the ultrasound diagnostic device 150 to perform the above processing, with reference to flow charts in FIG. 12 to FIG. 16.

FIG. 12 is a flow chart showing a processing procedure of examination report creation.

Firstly in Step S201, processing is performed in synchronization with an operation of a first process performed by an operator. Specifically, the B-mode image generation unit 104 generates a set of transverse images by the operator performing scanning in the longitudinal direction of a blood vessel. After the transverse images are generated, the image analysis measurement unit 105 calculates a plurality of IMTs characterizing cross-sectional shapes of the blood vessel, based on a contour of a lumen-intima interface and a contour of a media-adventitia interface shown in each of the transverse images in Step S202.

After the IMTs are calculated, the schematic diagram generation unit 106 generates a schematic diagram of the blood vessel in Step S203.

Next in Step S204, the B-mode image generation unit 104 generates a longitudinal image by the operator applying the probe 101 against a subject in a direction substantially perpendicular to the scanning direction of the scanning performed in Step S201.

In Step S205, the image analysis measurement unit 105 analyzes the longitudinal image, which is generated in Step S204, to obtain a diagnostic value of a diagnosed part. In Step S206, the relative position calculation unit 107 generates relative position information indicating a relative position of the diagnosed part shown in the longitudinal image. In Step S207, the measured data superimposing unit 108 generate an image for examination report by superimposing data of the diagnostic value on the schematic diagram, and causes the display unit 111 to display the generated image.

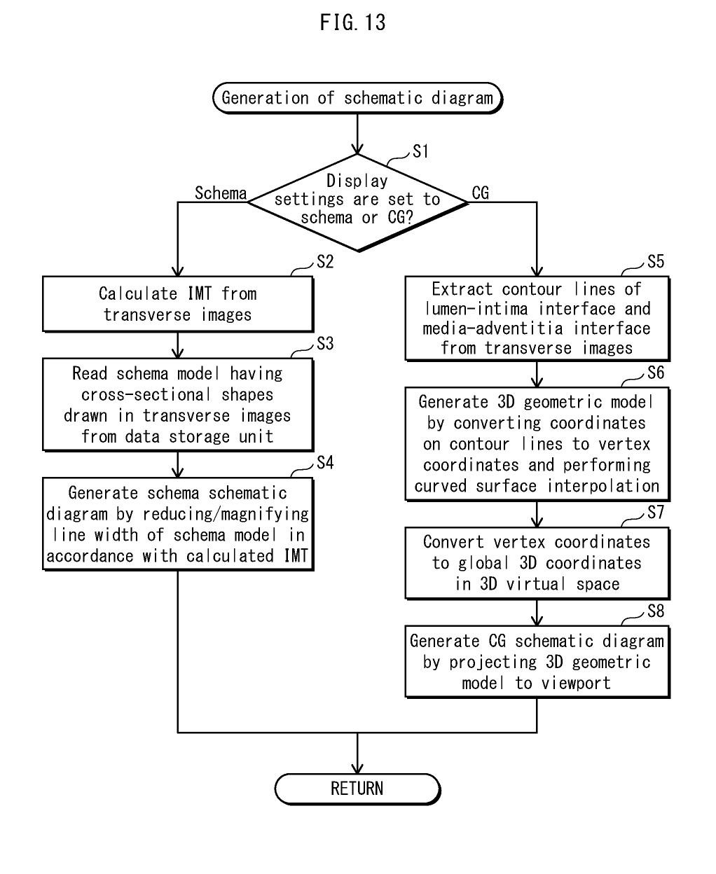

FIG. 13 is a flow chart showing a processing procedure of schematic diagram generation. In Step S1, judgment is made as to whether display settings of a window that is a display region are set to schema or CG When the display settings are set to schema in Step S1, IMT is calculated from a set of transverse images in Step S2, and a schema model having cross-sectional shapes drawn in the transverse images is read from the data storage unit 110 in Step S3. In Step S4, a schema schematic diagram is generated by reducing or magnifying a line width in the schema model in accordance with the calculated IMT.

When the display settings are set to CG in Step S1, a contour line of a lumen-intima interface and a contour line of a media-adventitia interface are extracted from the transverse images in Step S5. In Step S6, a 3D geometric model is generated by converting coordinates on the contour lines to vertex coordinates and performing curved surface interpolation between the vertex coordinates. Then, the schematic diagram generation unit 106 converts the vertex coordinates to global 3D coordinates in a 3D virtual space in Step S7, and projects the 3D geometric model to a viewport to obtain a CG schematic diagram in Step S8.

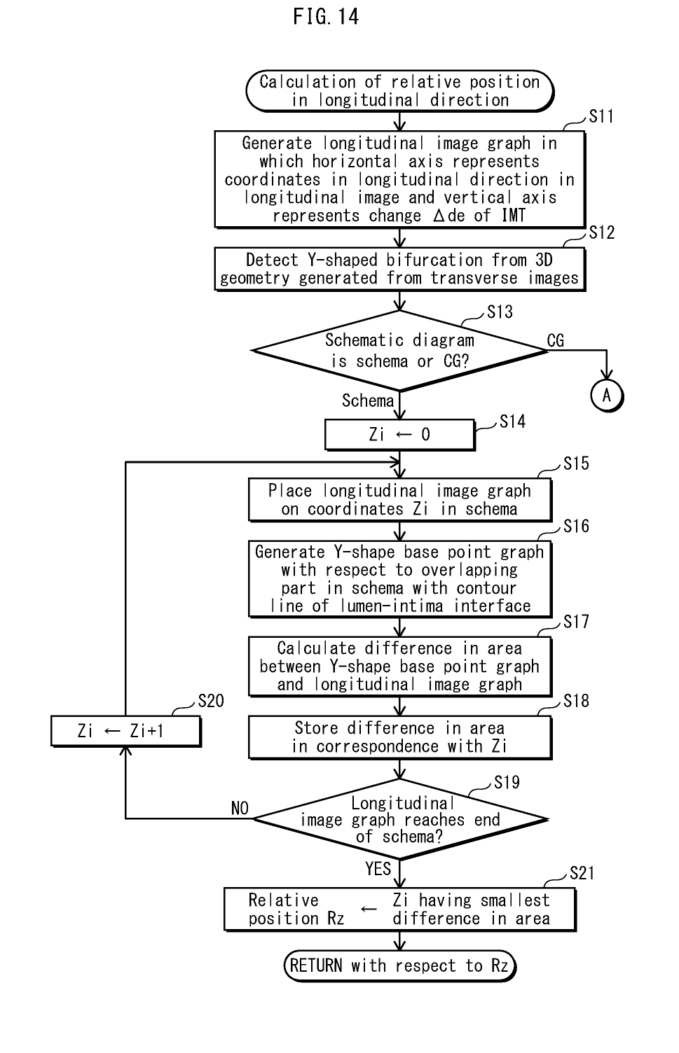

FIG. 14 is a flow chart showing a processing procedure of relative position calculation in the longitudinal direction. In Step S11, the relative position calculation unit 107 generates a longitudinal image graph in which the horizontal axis represents relative coordinates in the longitudinal direction in a longitudinal image, and the vertical axis represents change Δde in IMT.

In Step S12, a Y-shaped bifurcation is detected from a 3D geometry generated from a set of transverse images. In Step S13, judgment is made as to whether a schematic diagram is a schema or a CG When the schematic diagram is a schema in Step S13, a variable Zi is initialized in Step S14. Here, the variable Zi is a variable representing relative Z-coordinates along the Z-axis having the Y-shaped bifurcation as the base point. Then, the flow proceeds to a loop of Steps S15 to S20.

In the loop of Steps S15 to S20, the following processing is repeated. The longitudinal image graph is placed on coordinates Zi in the schema (Step S15). A Y-shape base point graph is generated with respect to an overlapping part in the schema that overlaps with the contour line of the lumen-intima interface shown in longitudinal image (Step S16). A difference in area between the Y-shape base point graph and the longitudinal image graph is calculated (Step S17). The difference in area is stored in correspondence with the coordinates Zi (Step S 18), and judgment is made as to whether the beginning of the longitudinal image graph reaches the end of the schema (Step S19). Until the judgment in Step S19 results in “YES”, the coordinates Zi are incremented (Step S20) and the flow returns to Step S15.

The loop of Steps S15 to S20 is repeated until the judgment in Step S19 results in “YES”. As a result, the difference in area between the longitudinal image graph and the Y-shape base point graph which is placed on each of a plurality of positions along the Z-axis is stored in correspondence with a value of coordinates Zi of the position. When the judgment in Step S19 results in “YES”, the flow proceeds to Step S21. In Step S21, one of the values of the coordinates Zi for which the smallest difference in area is calculated is selected, and the selected value of the coordinates Zi is stored as the relative position Rz.

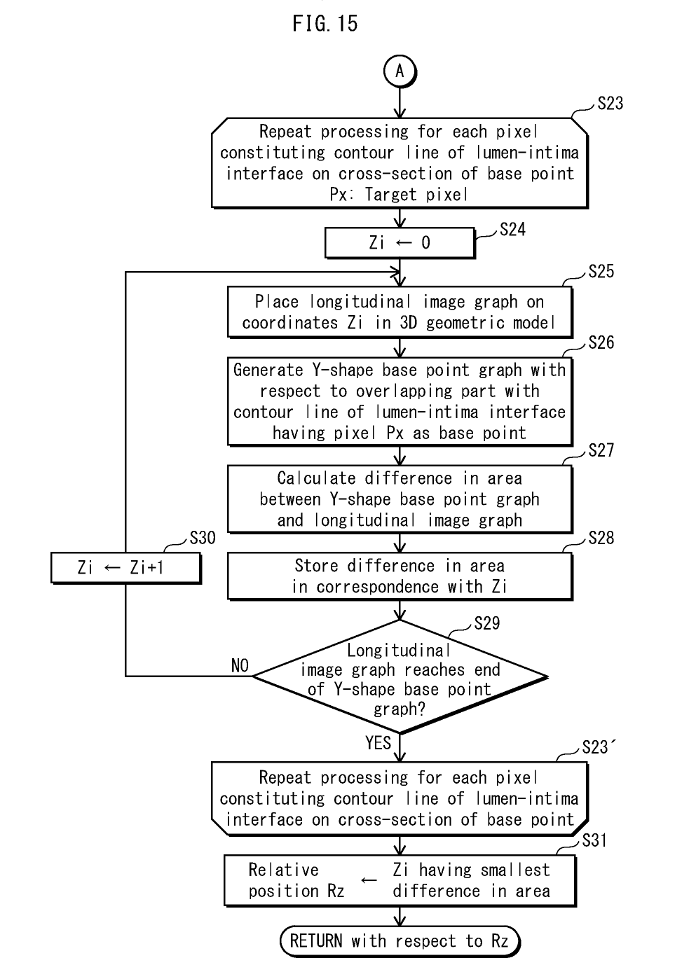

When the schematic diagram is a CG in Step S13, the flow proceeds to Step S23 in FIG. 15.

In Step S23 in combination with Step S23′, a loop is defined in which processing of Steps S24 to S30 is repeated for each of the pixels constituting the contour line of the lumen-intima interface on a cross-section of the Y-shaped bifurcation. These pixels that are targets for the processing in the loop are referred to as pixels Px.

In Step S24, a variable Zi is initialized. Here, the variable Zi is a variable representing relative Z-coordinates along the Z-axis having the Y-shaped bifurcation as the base point. Then, the flow proceeds to the loop of Steps S25 to S30. In the loop of Steps S25 to S30, the following processing is repeated. The longitudinal image graph is placed on coordinates Zi in the 3D geometric model (Step S25). A Y-shape base point graph is generated with respect to an overlapping part that overlaps with the closed curve representing the contour of the lumen-intima interface that has the pixel Px on the (Step S26). A difference in area between the Y-shape base point graph and the longitudinal image graph is calculated (Step S27). The difference in area is stored in correspondence with the coordinates Zi (Step S28), and judgment is made as to whether the beginning of the longitudinal image graph reaches the end of the Y-shape base point graph (Step S29). Until the judgment in Step S29 results in “YES”, the coordinates Zi are incremented (Step S30) and the flow returns to Step S25. As a result of the repetition, the difference in area between the longitudinal image graph and the Y-shape base point graph which is placed on each of a plurality of positions along the Z-axis is stored in correspondence with a value of the coordinates Zi of the position. When the judgment in Step S29 results in “YES”, the processing is complete with respect to one contour of the lumen-intima interface. When the processing of Steps S24 to S30 is complete with respect to all the pixels constituting each of the contour lines of the lumen-intima interfaces, the flow proceeds to Step S31. In Step S31, one of the values of the coordinates Zi for which the smallest difference in area is calculated is selected, and the selected value of the coordinates Zi is stored as the relative position Rz.

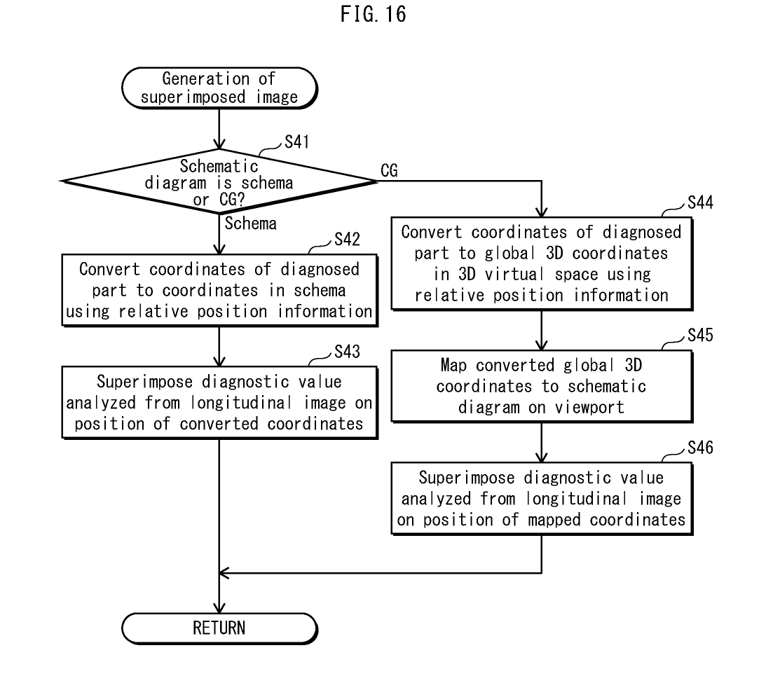

FIG. 16 is a flow chart showing a processing procedure of superimposed image generation. In Step S41, judgment is made as to whether a schematic diagram is a schema or a CG When the schematic diagram is a schema in Step S41, coordinates of the diagnosed part shown in the longitudinal image are converted to coordinates in the schema schematic diagram using the relative position information in Step S42. Then, data of the diagnostic value, which is analyzed from the longitudinal image, is superimposed on a position of the converted coordinates in the schema schematic diagram in Step S43.

When the schematic diagram is a CG in Step S41, the coordinates of the diagnostic part shown in the longitudinal image are converted to global 3D coordinates in the 3D virtual space using the relative position information in Step S44. In Step S45, vertex coordinates of the 3D geometric model in the 3D virtual space obtained by conversion are mapped to the viewport. Then in Step S46, data of the diagnostic value analyzed from the longitudinal image is superimposed on a position in the CG schematic diagram where the vertex coordinates are mapped.

According to the present embodiment as described above, the data of the diagnostic value is superimposed on the assumption that a position in the 3D geometric model is searched for which corresponds to the local part of the diagnosed part shown in the longitudinal image. Accordingly, it is possible to recognize the position of the diagnosed part such as a plaque exists in the entire blood vessel. This allows simple and easy creation of an examination report representing the position of the diagnostic part for which the diagnostic value is measured, without high-precision position detection by the probe.

Since the transverse images in the longitudinal direction correspond to the discontinuous Z-coordinates, the respective cross-sectional shapes shown in the transverse images are also discontinuous and lack smooth continuity. Furthermore, since scanning by the probe is manually performed, the cross-sectional shapes are discontinuous at irregular intervals. According to Embodiment 1, however, a position of a part on the contour line of the lumen-intima interface shown in the 3D geometric model is searched for, which has the same change in IMT with the plaque part shown in the longitudinal image. Therefore, even in the case where the position of the plaque part corresponds to a cross-sectional shape that is not included in the cross-sectional shapes shown in the transverse images, it is possible to identify a rough position of the plaque part in the blood vessel shown in the schema or the CG

The following describes modifications of Embodiment 1.

(Use of Histogram of Change Curve)

The above description has been given on the case where the area under the graph is used for searching for the most similar position. Alternatively, a histogram of a change curve also can be used for searching for the most similar position. The following describes the case where the most similar position is searched for using the histogram. In this case, a longitudinal image histogram and a Y-shape base point histogram are generated. In the longitudinal image histogram, the horizontal axis represents change Δde in IMT in a change curve, and the vertical axis represents a frequency of the change Δde in the change curve. In the Y-shape base point histogram, the horizontal axis represents change Δds in IMT in the overlapping part, and the vertical axis represents a frequency of the change Δds in a change curve. Comparison is performed between the longitudinal image histogram and the Y-shape base point histogram, thereby to search the Z-coordinates along the-Z axis for coordinates at which the frequency of the change is the largest is coincident between the longitudinal image histogram and the Y-shape base point histogram. The Z-coordinates which are found are identified as the relative position.

(Superimposing on Part in which Blood Flow Velocity is Measured)

Data of blood flow velocity may be superimposed, as the diagnostic value, on the schema or CG schematic diagram. Since the longitudinal image is obtained during measurement of the blood flow velocity, the relative position should be calculated as follows: (1) the relative position is calculated from the IMTs; (2) the longitudinal image is analyzed to obtain the blood flow velocity as the diagnostic value, and (3) data of the blood flow velocity as the diagnostic value is superimposed on a superimposing position in the schema or CG schematic diagram.

Here, the longitudinal image which is a target for obtaining the diagnostic value is a color Doppler image. A color Doppler image is an image in which blood flow velocity in each part is represented by color values. A target for relative position calculation is a part that corresponds to high blood flow velocity represented by color values in the color Doppler image.

(Superimposing on Part in which Elastic Properties are Measured)

Data of vascular elastic properties may be superimposed, as the diagnostic value, on the schema or CG schematic diagram.

Since the longitudinal image is obtained during measurement of vascular elastic properties, the relative position should be calculated as follows: (1) the relative position is calculated from the IMTs; (2) the longitudinal image is analyzed to obtain the vascular elastic properties as the diagnostic value, and (3) data of the vascular elastic properties as the diagnostic value is superimposed on a superimposing position in the schema or CG schematic diagram.

The vascular elastic properties are detected from a set of slow-scanning transverse images that are obtained by slow scanning in the longitudinal direction. The slow-scanning transverse images are a set of transverse images that are obtained within a single heartbeat by slowly moving the probe in the longitudinal direction. Variation in the blood vessel within the single heartbeat is analyzed from these slow-scanning transverse images, thereby to calculate a terminal diastole phase in which the blood vessel is the thickest within the single heartbeat and a systole phase in which the blood vessel is the thinnest within the single heartbeat.

The blood vessel has a part having a high elastic modulus in which a large difference in IMT exists between the terminal diastole phase and the systole phase. The blood vessel also has a part having a low elastic modulus in which little difference in IMT exists between the terminal diastole phase and the systole phase. Therefore, by dividing the highest value of change in IMT during the systole phase by the IMT in the terminal diastole phase to calculate a distortion amount in the radial direction of the blood vessel, it is possible to obtain the vascular elastic properties.

### Embodiment 2

In Embodiment 1, the relative position information is calculated from the transverse images, and the data of the diagnostic value analyzed from the longitudinal image is superimposed on the schematic diagram based on the relative position information. However, there is a possibility that the relative position information is wrong, and the display unit 111 displays the diagnostic value on a position which is shifted from the operator's intended position.

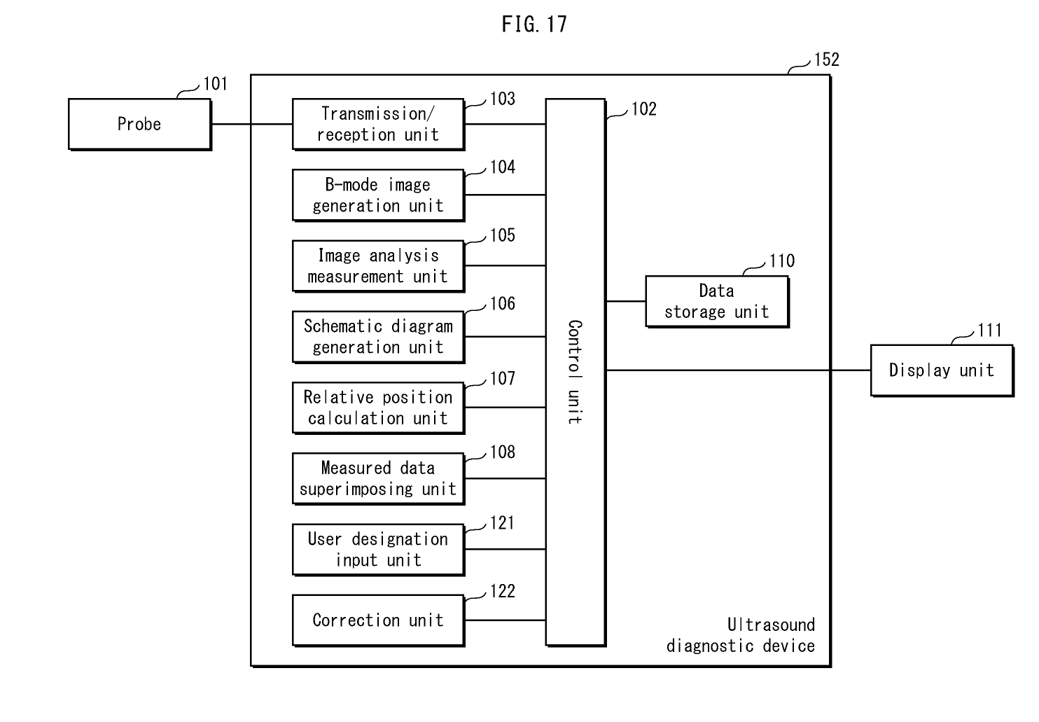

FIG. 17 is a block diagram showing an internal structure of an ultrasound diagnostic device 152 relating to Embodiment 2. The ultrasound diagnostic device 152 shown in FIG. 17 is different from the ultrasound diagnostic device 150 shown in FIG. 4, in terms of additionally including constituent elements which are not shown in FIG. 4, namely, a user designation input unit 121 and a correction unit 122.

The user designation input unit 121 receives a correction request on a schematic diagram from the operator by receiving an input from the pointing device.

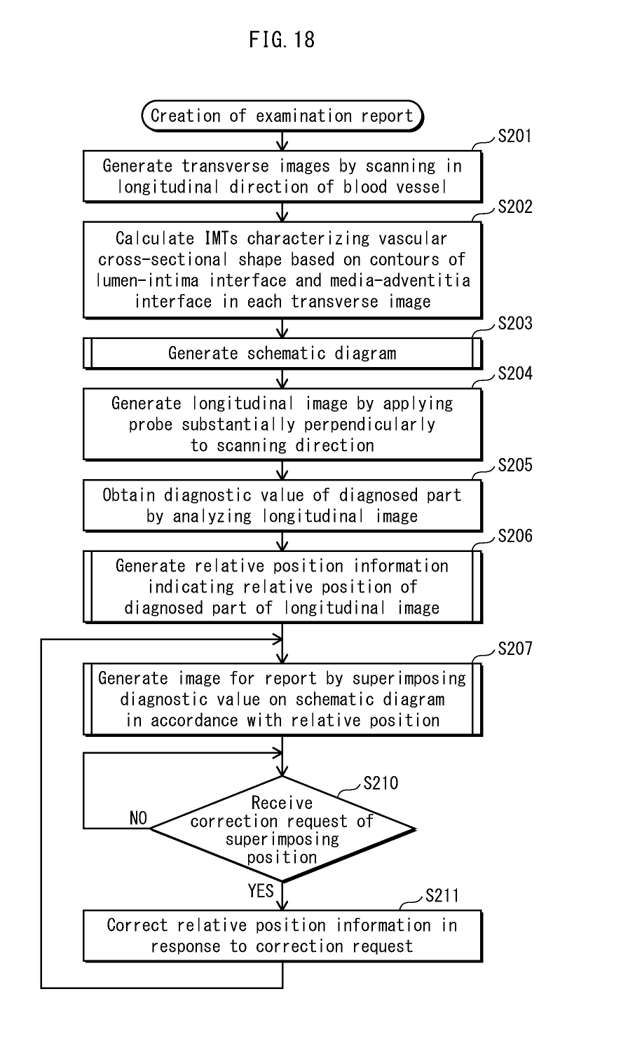

The correction unit 122 requests the relative position calculation unit 107 to correct the relative position information in response to the correction request received by the user designation input unit 121. The following describes the technical significance of the correction unit 122 being included in the ultrasound diagnostic device 152. In Embodiment 1, the superimposing position in the schematic diagram that corresponds to the position of the diagnosed part shown in the longitudinal image is identified by analyzing the transverse images. However, in the case where results of the analysis are wrong, it is impossible to precisely identify the superimposing position. There is a case where when a diagnostic target includes a plurality hyperplasia portions having the same thickness for example, a relative position cannot be uniquely identified and this results in wrong calculation of a superimposing position. In this way, in the case where results of analysis are wrong, it is impossible to precisely generate an image for examination report. In order to precisely generate an image for examination report even if results of analysis are wrong, the correction unit 122 is provided for correcting the relative position information in response to a correction request received by the user designation input unit 121. This completes the description of the ultrasound diagnostic device 152 relating to Embodiment 2. The following describes a processing procedure of examination report creation in Embodiment 2. FIG. 18 is a flow chart showing the processing procedure of examination report creation in Embodiment 2. The flow chart in FIG. 18 has Steps S210 and S211 in addition to the steps included in the flow chart in FIG. 12. The following describes these Steps which are newly added.

In Step S210, input of a correction request of a superimposing position is received from an operator.

In Step S211, the relative position information is corrected in response to the correction request received in Step S210. Then, the flow returns to Step S207, and the data of the diagnostic value is superimposed on the schematic diagram based on the corrected relative position information. In this way, an image for examination report is again generated. Assume a case where the operator feels that the superimposing position of the diagnostic value displayed by the display unit 111 is not appropriate. In this case, the operator performs operations of shifting the CG or the schema included in the examination report leftward, rightward, upward, and/or downward via interactive manipulation using the pointing device. In accordance with the operations, the relative position information is corrected and the data of the diagnostic value is superimposed on the schematic diagram based on the corrected relative position information. This allows prompt creation of an examination report which satisfies the operator.

According to the above structure, even in the case where the relative position is wrongly calculated due to difficulty in analysis of hyperplasia portions, it is possible to correct the relative position, thereby to further precisely create an image for examination report which reflects the operator's judgment.

### Embodiment 3

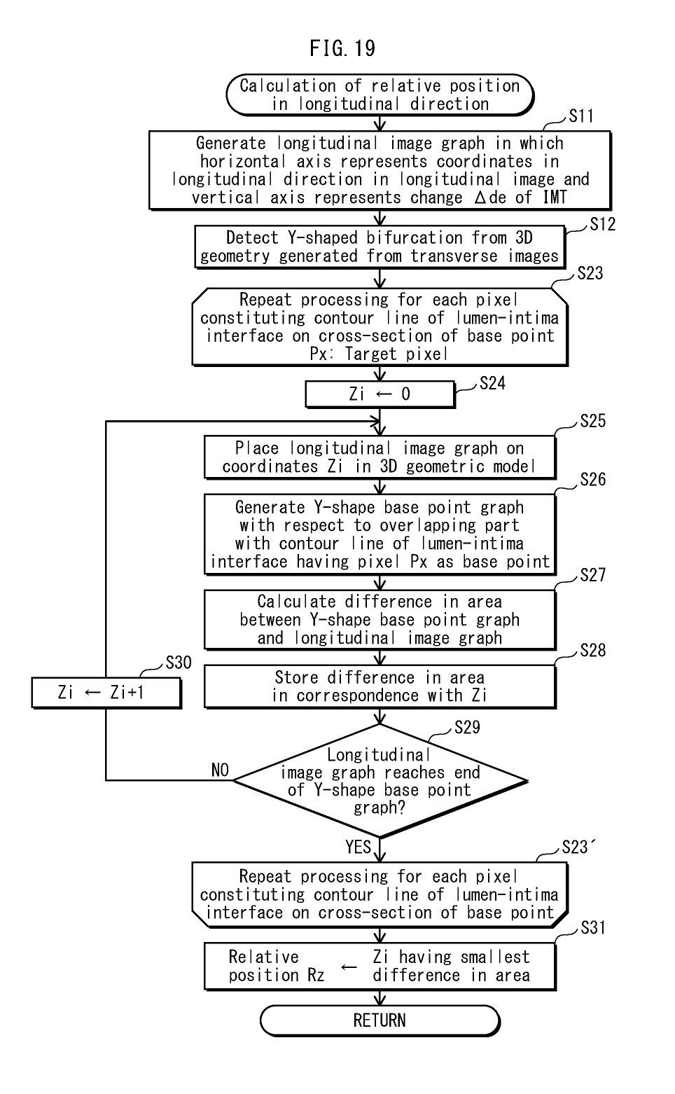

In Embodiment 1, in the case where a CG schematic diagram is generated, relative position information is generated based on a 3D geometric model. In the present embodiment compared with this, also in the case where a schema schematic diagram is generated, relative position information is generated based on a 3D geometric model. FIG. 19 is a flow chart showing a processing procedure of relative position calculation based on a 3D geometric model in the longitudinal direction in Embodiment 3. Since the flow chart in FIG. 19 is based on the flow charts in FIG. 14 and FIG. 15, steps in FIG. 19 that are the same as the steps in FIG. 14 and FIG. 15 have the same reference signs.

In the flow chart FIG. 19, a longitudinal image graph is generated in which the horizontal axis represents coordinates in the longitudinal direction in the longitudinal image, and the vertical axis represents the change Δde in IMT. In Step S12, a Y-shaped bifurcation is detected from a 3D geometry generated from the transverse images. Then, the flow proceeds to a loop defined by Step S23 in combination with Step S23′ in which processing of Steps S25 to S30 is repeated for each of the pixels constituting the contour line of the lumen-intima interface on the cross-section of the Y-shaped bifurcation. These pixels that are targets for the processing in the loop are referred to as pixels Px.

In Step S24, a variable Zi is initialized. Here, the variable Zi is a variable representing relative Z-coordinates along the Z-axis having the Y-shaped bifurcation as the base point. Then, the flow proceeds to a loop of Step S25 to S30. In the loop of Steps S25 to S30, the following processing is repeated. The longitudinal image graph is placed on coordinates Zi in the 3D geometric model (Step S25). A Y-shape base point graph is generated with respect to an overlapping part that overlaps with a contour of the lumen-intima interface that has the pixel Px on a closed curve representing the contour of the lumen-intima interface (Step S26). A difference in area between the Y-shape base point graph and the longitudinal image graph is calculated (Step S27). The difference in area is stored in correspondence with the coordinates Zi (Step S28), and judgment is made as to whether the beginning of the longitudinal image graph reaches the end of the Y-shape base point graph (Step S29). Until the judgment in Step S29 results in “YES”, the coordinates Zi are incremented (Step S30) and the flow returns to Step S25. As a result of the repetition, the difference in area between the longitudinal image graph and the Y-shape base point graph which is placed on each of a plurality of positions along the Z-axis is stored in correspondence with a value of the coordinates Zi of the position. When the judgment in Step S29 results in “YES”, the processing is complete with respect to one contour of the lumen-intima interface. When the processing of Steps S24 to S30 is complete with respect to all the pixels constituting each of the contour lines of the lumen-intima interfaces, the flow proceeds to Step S31. In Step S31, one of the values of the coordinates Zi that corresponds to the smallest difference in area is selected, and the selected value of the coordinates Zi is stored as the relative position Rz. As a result, one of the values of the Z-coordinates in the longitudinal direction of the blood vessel that corresponds to the smallest difference in area between the Y-shape base point graph and the longitudinal image graph is identified as the relative position. In this way, the coordinates Rz as the relative coordinates in the Z-axis direction are determined. Then in Step S42 in FIG. 16 shown in Embodiment 1, the coordinates of the diagnostic part are converted to coordinates on the schema schematic diagram using the relative position information, and in Step S43, the data of the diagnostic value analyzed from the longitudinal image is superimposed on the position of the converted coordinates.

This allows superimposing of the data of the diagnostic value on the schema schematic diagram based on the relative position information which is precise, in addition to the case of the CG schematic diagram.

<Remarks>

Although the above description has been provided on the most preferred embodiments known to the applicant at the time of filing the application, further improvement and modification may be performed on the following technical topics.

(Generation of Transverse images and Longitudinal Image)

In Embodiment 1, transverse images and a longitudinal image are respectively generated by ultrasound scanning through the first process and the second process. Alternatively, the transverse images and the longitudinal image may be respectively generated by consecutive scanning in the transverse direction and in the longitudinal direction.

(Target for Overlapping Longitudinal Image Graph)

A contour line of a blood vessel generated by transverse scanning often has a front wall and a back wall but lacks a side wall for the following reason. While the front wall and the back walls are drawn because of being perpendicular to ultrasound and reflecting the ultrasound, the side wall tends not to be drawn because of being parallel to the ultrasound and difficult to reflect the ultrasound. Therefore, it is desirable to overlap the longitudinal image graph with the Y-shape base point graph of each of the front wall and the back wall which ultrasound reflects to search for a position in which a difference in area is the smallest as the relative position.

(Moving Direction of Probe)

The above description has been given exemplifying the case in which a medical worker linearly moves the ultrasound probe on the subject to sequentially obtain reflected ultrasound. However, the movement of the ultrasound probe is not limited to linear movement. More specifically, the description is also applicable to the case in which an operator moves the ultrasound probe curvilinearly.

(Necessity of Data Storage Unit)

Embodiment 1 is characterized by the relative position calculation method of calculating a superimposing position in a schematic diagram that corresponds to a position of a diagnostic part. Therefore, whether or not to provide the ultrasound diagnostic device 150 with the data storage unit 110 is arbitrary.

(Variation of Ultrasound Diagnostic Device 150)

The probe 101 and the display unit 111 may be included in the ultrasound diagnostic device 150. Alternatively, the probe 101 and the display unit 111 do not need to exist. This is because it is only necessary that an ultrasound signal which is input by the probe 101 is processed by the ultrasound diagnostic device 150, and it is only necessary that a video signal indicating a schematic diagram on which data of a diagnostic value is superimposed is output by the ultrasound diagnostic device 150 and the output video signal is displayed by the display unit 111. Part or all of the processing units included in the ultrasound diagnostic device relating to the above embodiments may be included in the probe 101.

(Variation of Probe 101)

The probe 101 may be a probe in which ultrasound transducers are arranged in a one-dimensional direction, or a two-dimensional array probe in which ultrasound transducers are arranged in a matrix.

(Identification of Media-Adventitia Interface and Lumen-Intima Interface)

A contour extraction technique may be used for identifying a media-adventitia interface and a lumen-intima interface. In this case, by extracting a contour of the media-adventitia interface and a contour of the lumen-intima interface, it is possible to measure a distance therebetween as IMT.

(Variation of Calculation of Relative Position Information)

Relative position information may be calculated by the following method according to which a part on a schematic diagram which having the local maximum IMT is extracted, the local maximum IMT is compared with a diagnostic value analyzed from a longitudinal image, and thereby to calculate a superimposing position in the schematic diagram that corresponds to a position of the diagnostic part shown in the longitudinal image.

(Circuit Integration)

The processing units included in the ultrasound diagnostic devices relating to the embodiments are each typically implemented as an LSI that is an integrated circuit. These processing units may be individually configured as single chips or may be configured such that a part or all of the processing units are included in a single chip. Furthermore, the method of circuit integration is not limited to LSIs, and implementation through a dedicated circuit or a general-purpose processor is also possible. A field programmable gate array (FPGA) that allows programming after LSI manufacturing or a reconfigurable processor that allows reconfiguration of connections and settings of circuit cells inside the LSI may also be used.

Furthermore, part or all of the functions of the ultrasound diagnostic devices relating to the embodiments may be implemented by a processor such as a CPU executing a program. In addition, the present invention may be the above program. Alternatively, the present invention may be a non-transitory computer-readable recording medium in which the program is recorded. Furthermore, it should be obvious that the program can also be distributed via a transmission medium such as the Internet.

(Combination of Functions)

At least part of the functions of the ultrasound diagnostic devices relating to the embodiments and the modifications thereof may be combined with each other. Furthermore, all the numerical figures used above are given as examples to specifically describe the present invention, and therefore the present invention is not limited by such illustrative numerical figures.

(Configuration of Functional Blocks)

Separation of the functional blocks in the block diagrams is merely an example, and plurality of functional blocks may be implemented as a single functional block, a single functional block may be separated into a plurality of functional blocks, or part of functions of a functional block may be transferred to another functional block. Furthermore, the functions of functional blocks having similar functions may be processed in parallel or in time division by single hardware or software.

(Execution Sequence of Steps)

The sequence in which the above steps are executed is given as an example to specifically describe the present invention, and therefore other sequences are possible. Furthermore, part of the above steps may be executed simultaneously (in parallel) with another step. Moreover, the present invention includes various types of modifications obtainable through modifications to the embodiments that may be conceived of by a person skilled in the art, as long as such modifications do not cause deviation from the general concept of the present invention.

Although the present invention has been fully described by way of examples with reference to the accompanying drawings, it is to be noted that various changes and modifications will be apparent to those skilled in the art. Therefore, unless such changes and modifications depart from the scope of the present invention, they should be construed as being included therein.

### INDUSTRIAL APPLICABILITY

The ultrasound diagnostic device relating to the present invention has a unit for identifying a relative positional relationship between a schematic image and an image for measurement, and is useful in diagnosis of arteriosclerosis.

What is claimed is:

1. An ultrasound diagnostic device that analyzes a plurality of ultrasound images that are obtained by ultrasound scanning on a diagnostic target, the ultrasound images including a set of material images for analyzing a shape of the diagnostic target and a diagnostic image for analyzing a diagnostic value of a specific part included in the diagnostic target, the ultrasound diagnostic device comprising:
a generation circuit that generates a schematic diagram showing a schematic shape of the diagnostic target based on the shape of the diagnostic target that is analyzed from the material images;
a calculation circuit that calculates a relative position of a local shape of the specific part shown in the diagnostic image with respect to the shape of the diagnostic target analyzed from the material images; and
a superimposing circuit that identifies a position on the schematic shape of the diagnostic target shown in the schematic diagram that corresponds to a position of the specific part based on the relative position, and superimposes data of the diagnostic value on the identified position on the schematic shape of the diagnostic target shown in the schematic diagram.

2. The ultrasound diagnostic device of claim 1, wherein
the calculation circuit overlaps the local shape shown in the diagnostic image on each of a plurality of positions on the shape analyzed from the material images, and calculates a degree of similarity in each of the overlapped positions between the local shape shown in the diagnostic image and the shape analyzed from the material images, and
the relative position is one of the overlapped positions for which a highest degree of similarity is calculated.

3. The ultrasound diagnostic device of claim 2, wherein
the diagnostic target is a blood vessel,
the shape analyzed from the material images is identified from vascular wall thicknesses that are each measured in a different position on a cross-section of the blood vessel, and
the degree of similarity is calculated from a difference in change value of a vascular wall thickness in a longitudinal direction of the blood vessel.

4. The ultrasound diagnostic device of claim 3, wherein
the calculation circuit generates a change curve that represents change in the vascular wall thickness in the longitudinal direction of the blood vessel, and
the difference in change value is a difference in area under the change curve in the overlapped positions between the shape analyzed from the material images and the local shape shown in the diagnostic image.

5. The ultrasound diagnostic device of claim 2, wherein
the calculation circuit generates a histogram of change in vascular wall thickness in a longitudinal direction of a blood vessel, and
the degree of similarity is a degree of coincidence in terms of the histogram between the shape analyzed from the material images and the local shape shown in the diagnostic image.

6. The ultrasound diagnostic device of claim 1, wherein
the diagnostic target is a blood vessel,
the material images each show a cross-sectional shape of the blood vessel on different coordinates along an axis in a longitudinal direction of the blood vessel, and
the schematic diagram is generated by performing interpolation between the respective cross-sectional shapes shown in the material images to generate a three-dimensional geometric model representing a three-dimensional geometry of the blood vessel in a three-dimensional virtual space, and projecting an image of the three-dimensional geometric model to a viewport in the three-dimensional virtual space.

7. The ultrasound diagnostic device of claim 1, wherein
the diagnostic target is a blood vessel,
the shape of the diagnostic target is identified from vascular wall thicknesses that are each measured in a different position on a cross-section of the blood vessel, and
the schematic diagram is a schema that represents a cross-sectional shape of the blood vessel, and is generated by correcting a schema model in accordance with the vascular wall thicknesses.

8. The ultrasound diagnostic device of claim 1, wherein
the blood vessel is a carotid artery, and
the calculation circuit detects a Y-shaped bifurcation from the local shape that is three-dimensionally shown in the diagnostic image, and determines the Y-shaped bifurcation as a base point of the relative position.

9. The ultrasound diagnostic device of claim 1, wherein
the diagnostic target is a blood vessel, and
the diagnostic value indicates vascular wall thickness.

10. The ultrasound diagnostic device of claim 1, wherein
the diagnostic target is a blood vessel, and
the diagnostic value indicates blood flow velocity.

11. The ultrasound diagnostic device of claim 1, wherein
the diagnostic target is a blood vessel,
the diagnostic value indicates elasticity of a vascular tissue.

12. The ultrasound diagnostic device of claim 1, wherein
the diagnostic image is a longitudinal cross-sectional image that is obtained by ultrasound scanning in a longitudinal direction of the diagnostic target, and
the material images are a plurality of transverse cross-sectional images that are obtained by a plurality of instances of ultrasound scanning in a transverse direction of the diagnostic target.

13. An ultrasound diagnostic method comprising:
obtaining a plurality of ultrasound images that are obtained by ultrasound scanning on a diagnostic target, the ultrasound images including a set of material images for analyzing a shape of the diagnostic target and a diagnostic image for analyzing a diagnostic value of a specific part included in the diagnostic target;
obtaining the diagnostic value of the specific part included in the diagnostic target;
generating a schematic diagram showing a schematic shape of the diagnostic target based on the shape of the diagnostic target that is analyzed from the material images;
calculating a relative position of a local shape of the specific part shown in the diagnostic image with respect to the shape of the diagnostic target analyzed from the material images; and
identifying a position on the schematic shape of the diagnostic target shown in the schematic diagram that corresponds to a position of the specific part based on the relative position, and superimposing data of the diagnostic value on the identified position on the schematic shape of the diagnostic target shown in the schematic diagram.

14. A non-transitory computer-readable recording medium having recorded therein a program for causing a computer to execute the ultrasound diagnostic method of claim 13.
